SEQUENCE LISTING (1 sequence)

SEQ ID NO: 1 - protein, 10 aa, synthetic construct
  NON_TER 10: note = Linker
GRKKRRQRRR

(19) United States
(12) Patent Application Publication
(10) Pub. No.: US 2026/0139276 A1
(43) Pub. Date: May 21, 2026

(54) METHOD AND AN APPARATUS FOR PENETRATING A CELL WITH A PARTICLE

(71) Applicant: AGEN FOR SCIENCE, TECHNOLOGY AND RESEARCH, Singapore (SG)

(72) Inventor: Zhi Wei Tay, Singapore (SG)

(21) Appl. No.: 19/114,725

(22) PCT Filed: Oct. 4, 2023
(86) PCT No.: PCT/SG2023/050667

§ 371 Date: Mar. 24, 2025

(30) Foreign Application Priority Data
Oct. 4, 2022 (SG) 10202251255E

(51) Int. Cl. C12N 15/89; C12M 1/32; C12M 1/42

(52) U.S. Cl. CPC C12N 15/895; C12M 23/12; C12M 35/02; C12M 35/04

(57) ABSTRACT

A method and apparatus for penetrating a cell with a particle are described. The method includes providing a layer of cells at or proximal to a bottom end of a vessel; adding a plurality of particles to the vessel; and applying a force to move the plurality of particles towards the bottom end of the vessel to cause at least some of the plurality of particles to penetrate at least some of the cells. The apparatus includes a vessel having a bottom end for containing a layer of cells at the bottom end; and a force exerting component configured to apply a force to move a plurality of magnetic particles within the vessel towards the bottom end of the vessel to penetrate at least some of the cells with at least some of the plurality of magnetic particles.

### REFERENCE TO RELATED APPLICATIONS

The present application claims priority to Singapore patent application number 10202251255E filed on 4 Oct. 2022 titled “Non-vehicular methods for efficient administration of magnetic labels into non-permissive cells” and which is incorporated by reference herein in its entirety.

### FIELD OF THE INVENTION

The present invention relates to the insertion of foreign particles into cells, and an apparatus for the insertion.

### BACKGROUND OF THE INVENTION

In vivo cell tracking is extremely relevant to many medical applications such as regenerative medicine (stem cell) implants and immunotherapy (adoptive cell transfer (ACT) or CAR T-cell therapy (CAR-T)). Magnetic labels have been shown to be one of the most long-lasting imaging labels for in vivo cell tracking as the signal does not radioactively decay over time (typically short half-lives of few hours) and does not undergo photobleaching or rapid digestion like optical dyes. However, existing methods of magnetic labelling of cells are limited by having a long labelling time of up to 24 hours and poor uptake for non-phagocytic cells which are used in ACT and CAR-T. The extended labelling time may be toxic for the primary cells being labelled leading to lower viability of the labelled cells. The sorting of the labelled cells is possible but expensive and with typically poor yield. Microfluidic approaches have been proposed but these are difficult to scale-up to accommodate the tens of millions of cells needed for clinical cell therapies. Vehicular methods using antibody-targeting or liposomal coating significantly increase the cost and complexity of the labelling process. Lewin et al. (Nature Biotechnology, 2000, 18 (4): 410-4, doi: 10.1038/74464) describes the synthesis of HIV Tat-peptide conjugated magnetic nanoparticles and a labelling protocol for uptake into CD34+ haemopoietic progenitor cells for in vivo imaging. However, the method described suffers the drawbacks of requiring high concentrations of the nanoparticles which results in a large majority being left behind in the media as waste. This is uneconomical and inefficient and will pose problems scaling up.

Walczak P et al. (Nanomedicine: Nanotechnology, Biology, and Medicine, 2006, 2 (2): 89-94, doi: 10.1016/j.nano.2006.01.003) describes the use of standard electroporation to enable membrane permeabilization for efficient uptake of magnetic nanoparticles into neural stem cells and leukocytes for cellular magnetic resonance imaging. However, the uptake efficiency is not very high (only 1-5 pg Fe per cell). Further increase of efficiency required higher voltage settings (1 kV or 2.5 kV/cm) in conjunction with a very short pulse but resulted in a high loss of viability (20%) which decreases the overall efficiency. Furthermore, the common electroporation apparatus widely available is suited only for suspension cells and high-throughput labelling with adherent cells requires a specialised setup.

Kim H S et al. (NMR in Biomedicine, 2010, 23:514-22, doi: 10.1002/nbm. 1487) describes an evaluation of labelling efficiency of three iron-oxide based MRI contrast agents by incubating target human mesenchymal stem cells with poly-L-lysine via a biochemical method.

CN111671921 describes a method of labelling cells with quantum dot-containing magnetic particles using virus-like particles. However, in CN111671951 the incubation time is longer and the micron-sized magnetic particles are very large and difficult to squeeze into smaller cells such as naïve T-lymphocytes (only 6 microns across with only 30% cytoplasmic volume to fit the magnetic particle). This fact is acknowledged by the authors which state that since the MPIO size is large and difficult to ingest by the cells via phagocytosis, the cells that can be labelled are limited and is mainly focused on macrophages and cells with phagocytosis functions (such as liver library Freund cells).

JP2009002685 describes a method of labelling of the surface of red blood cells with magnetic nanoparticles using a custom gelatin gum carrier.

KR1020090110076 describes a method of labelling target cells with nanoparticles in a perfluorocarbon nanoemulsion and packaging the particles into liposomes in magnetic resonance imaging of the cells.

The existing methods described above are generally either not efficient or limited to specific cell types, and these limitations prevent the widespread adaption of magnetic labelling.

### SUMMARY OF THE INVENTION

In a first aspect, there is provided a method of penetrating a cell with a particle, the method comprising: providing a layer of cells at or proximal to a bottom end of a vessel; adding a plurality of particles to the vessel; and applying a force to move the plurality of particles towards the bottom end of the vessel to cause at least some of the plurality of particles to penetrate at least some of the cells.

In a second aspect, there is provided an apparatus for penetrating a cell with a magnetic particle. The apparatus comprising a vessel having a bottom end for containing a layer of cells at the bottom end; and a force exerting component configured to apply a force to move a plurality of magnetic particles within the vessel towards the bottom end of the vessel to penetrate at least some of the cells with at least some of the plurality of magnetic particles.

Advantageously, the method and apparatus allow for a lower concentration of particles including magnetic nanoparticles to be used in the labelling leading to significantly less wastage of the particles. It is well-known that when co-incubating nanoparticles in culture media, the large majority of nanoparticles are still left in the media, yet using lower concentrations result in poorer labelling efficiency. The method and apparatus described herein effectively achieves high concentrations with low amounts of nanoparticles.

### BRIEF DESCRIPTION OF THE DRAWINGS

Figure (FIG. 1 shows the comparison of conventional magnetic labelling of cells which is passive incubation of magnetic label with the cells in media (left) to the proposed method of using magneto-mechanical forces to enable label penetration into the cell (right). The top part of FIG. 1 shows the combination of the magnetic nanoparticles (MNPs) and cells. Centrifugal forces 20 are applied first to layer the cells at the bottom of the V or U well, then magnetic forces 15 are applied to form a sandwich of the cells.

FIG. 2 shows the magnetic nanoparticle (MNP) uptake efficiency for the centrifuge sandwich method (1.5-2.5 hour spin). The results show that increasing the nanoparticle concentration in media does not improve labelling efficiency, suggesting that centrifugation causes migration of nanoparticles towards bottom of well, reaching a much higher effective concentration that plateaus and thus any further increasing in media concentration of nanoparticles has no effect.

FIG. 3 shows the magnetic nanoparticle uptake efficiency for the Focal Magnetic Plate sandwich method (with 3 hour incubation). The positive sign and negative sign respectively indicate whether the magnet or Tat peptide is present or absent. The results show that the Focal Magnet increases the uptake significantly over Tat peptide alone, and thus further improves the uptake above what Tat peptide performs.

FIG. 4 shows a schematic design of the magnetic array specifically optimized for the magnet sandwich mechanism as it maximizes vertical pull force at the centre of the U or V bottom of each of the 96 wells and minimizes vertical pull force everywhere else, ensuring that nanoparticles are not wasted on other regions of the 96 well with lower cell density. The field lines at the edge of the 96 well are designed to flow horizontally such that nanoparticles are not pulled to the edges of the well but rather to the centre of the well. Lastly, the design maximizes safety as there is only a strong pull force within 1 to 2 cm of the plate and there is essentially no pull beyond this distance due to redirection of flux lines into neighbouring magnets rather than allowing them to extend out.

FIG. 5 shows a graphical display of the magnetic field and magnetic foci at each well centre.

FIG. 6 shows a graphical display of the magnetic field of a commercial magnet slab.

FIG. 7 shows three pictures of the centrifugal and magnetic concentration of the magnetic nanoparticles and cells, from left to right, the pictures are before the concentration, at 90 minutes of the concentration process, and at the end of the process.

### DETAILED DESCRIPTION OF THE INVENTION

The following detailed description is merely exemplary in nature and is not intended to limit the invention or the application and uses of the invention. Furthermore, there is no intention to be bound by any theory presented in the preceding background of the invention or the following detailed description.

Other technical advantages may become readily apparent to one of ordinary skill in the art after review of the following figures and description.

Features that are described in the context of an embodiment may correspondingly be applicable to the same or similar features in the other embodiments. Features that are described in the context of an embodiment may correspondingly be applicable to the other embodiments, even if not explicitly described in these other embodiments. Furthermore, additions and/or combinations and/or alternatives as described for a feature in the context of an embodiment may correspondingly be applicable to the same or similar feature in the other embodiments.

Modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set.

As used herein, the articles “a”, “an” and “the” as used with regard to a feature or element include a reference to one or more of the features or elements. As used herein, the term “and/or” includes any and all combinations of one or more of the associated listed items. As used herein, the terms “first,” “second,” and “third,” etc. are used merely as labels, and are not intended to impose numerical requirements on their objects. As used herein, the terms “top”, “bottom”, “left”, “right”, “side”, “vertical” and “horizontal” are used to describe relative arrangements of the elements and features. As used herein, the term “each other” denotes a reciprocal relation between two or more objects, depending on the number of objects involved.

Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the invention. Where a value being discussed has inherent limits, for example where a component can be present at a concentration of from 0 to 100%, or where the pH of an aqueous solution can range from 1 to 14, those inherent limits are specifically disclosed. Where a value is explicitly recited, it is to be understood that values which are about the same quantity or amount as the recited value are also within the scope of the invention, as are ranges based thereon.

Described herein are new and facile non-vehicular methods that are able to magnetically label cells efficiently and cheaply with widely available 96 well plates. Plates with other number of wells may also be used. A high throughput of tens of millions of cells may be achieved with the custom designed spin protocol and magnetic device described herein.

Described is a method of penetrating a cell with a particle. The method comprising: providing a layer of cells at or proximal to a bottom end of a vessel; adding a plurality of particles to the vessel; and applying a force to move the plurality of particles towards the bottom end of the vessel to cause at least some of the plurality of particles to penetrate at least some of the cells.

Preferably, the bottom end of the vessel is tapered. In an embodiment, the bottom end has a U-shaped or a V-shaped vertical cross-section. Advantageously, the tapered bottom helps to align the cell physical location focus with the magnetic focus or focal point. Further, the tapered bottom end increases the surface area to volume ratio compared to a flat bottom. The tapered bottom end concentrates the particles and cells and enables the particles to penetrate the cells more efficiently.

In an embodiment, the force includes a magnetic force exerted by a magnetic field that increases from a top region of the vessel to the bottom end of the vessel, the top region being opposite to the bottom end, and wherein the plurality of particles are magnetic particles. This may be termed a magnetic sandwich method as the cells are sandwiched between the magnetic particles and the bottom of the vessel. The increasing magnetic field thus provides a magnetic field gradient which is defined as the change in magnetic field with distance. Preferably, the magnetic field increases exponentially from the top region of the vessel to the bottom end of the vessel. The magnetic force action on the magnetic particles is proportional to the magnetic field gradient thus an exponential increase translates to a significantly larger magnetic force acting on the particles at the bottom of the vessel.

Preferably, the magnetic force is a significant magnetic force in three axes that concentrates the plurality of particles towards the bottom end of the vessel wherein one of the three axes is parallel to a longitudinal axis of the vessel, and the three axes are spatially orthogonal to one another. More preferably, the magnetic field that increases from the top region of the vessel to the bottom end of the vessel is greater than 5 milliTesla per millimetre. Advantageously, this creates a magnetic focal point in three axes that accumulate the magnetic particles in a three axes focus region at the bottom end of the vessel. The magnetic focal point design provides significantly improved efficiency as opposed to a generic vertical force (magnetic or gravitational) acting on the magnetic particles to concentrate the particles to the bottom of the vessel.

Preferably, the magnetic force is generated by one magnet of a plurality of magnets in a magnetic array, wherein the plurality of magnets is arranged in an alternating polarity configuration.

Preferably, at least one of the following conditions are fulfilled: (i) the magnetic force is applied for at least 5 minutes, more preferably at most 12 hours, even more preferably the magnetic force is applied from 2 to 3 hours; and (ii) the magnetic force is applied in the absence of ethylenediaminetetraacetic acid (EDTA) or a salt thereof (a salt of EDTA). More preferably, both conditions are fulfilled. The absence of EDTA improves the cell viability and is atypical in magnetic labelling as EDTA prevents clumping in magnetic nanoparticle suspensions.

In an embodiment, the plurality of particles comprises iron oxide particles, preferably the iron oxide particles are contained within a dextran shell. The dextran shell or shell in general may be functionalised with a cell-penetrating peptide or fluorescent dye. Preferably, at least one of the following conditions are fulfilled: (i), the method provides an iron uptake of at least 1 picogram iron per cell; and (ii) the plurality of particles is present at a concentration of at least 200 μg per mL, in particular when the particles are magnetic particles.

In an embodiment, the force comprises a centrifugal force in combination with the magnetic force.

In an embodiment, the force comprises a centrifugal force. Preferably, the force is the centrifugal force. Advantageously, this may be used with non-magnetic particles. In other words, the force is a centrifugal force alone or in combination with the magnetic force. Thus, the centrifugal force may be used by itself in what may be termed a centrifugal sandwich method or used in combination with the magnetic sandwich method.

Preferably, applying the centrifugal force comprises centrifuging the cells for at least 1 hour, preferably at most 3 hours.

In an embodiment, the layer of cells is present at a concentration of at least 100,000 cells per vessel.

In an embodiment, the particles have higher density than the cells, preferably the particles have at least 10 times higher density than the cells, more preferably the particles have at least 50 times higher density than the cells, even more preferably the particles have at least 100 times higher density than the cells.

In an embodiment, the vessel is a well of a multi-well plate, preferably a well of a 96-well plate. Preferably, the magnetic array may also be placed in a plate with the same number of wells. In an embodiment, the plate for the cells have a tapered bottom end whereas the plate for the magnetic array has a flat bottom end. Advantageously, this approach utilises widely available 96 well plates that accommodate both suspension and adherent cells which allow for high-throughput screening at low costs.

Preferably, comprising providing a layer of cells at or proximal to a bottom end of a vessel comprises centrifuging the cells to provide the layer of cells at or proximal to the bottom end of the vessel. It should be noted that the centrifugal force to provide the layer of cells is applied separately from the centrifugal force to allow the particles to penetrate the cells.

Preferably, the layer is a single layer only, preferably a dense cellular single layer. In the context of various embodiments, the single layer of cells refers to a monolayer of cells without any additional layer of cells. A calibration process, which takes into account the amount of cells in the vessel and the floor surface area, is used to ensure that a monolayer of cells is provided upon centrifuging. The calculation of the number of cells may be as follows:

$$Number of cells = \frac{( measured floor area )}{π * {( cell radius )}^{2}}$$

For example, a 6 micron diameter cell for a floor area of 1.5 mm×1.5 mm requires approximately 80,000 cells to form a dense monolayer.

In an embodiment, the method comprises adding a delivery vehicle to increase the penetration of the particles into the cells. The delivery vehicle may be selected from the group consisting of antibodies, liposomes, ligands and cell-penetrating peptides. In an example, the delivery vehicle is a cell-penetrating peptide. An example of a cell-penetrating peptide is a Tat peptide comprising GRKKRRQRRR (SEQ ID NO:1) and a linker, the linker comprises any suitable functional groups that allow linking and conjugation. The Tat peptide may be GRKKRRQRRR-linker, where the dash represents a covalent bond. The linker may be bonded to the end arginine amino acid (R). Preferably, the linker comprises amino acids.

In an embodiment, a delivery vehicle is absent from the vessel.

In an embodiment, the cells have an innate low uptake of the particles. The cells may be selected from the group consisting of lymphocytes, neutrophils, monocytes, macrophages and stem cells. Preferably, the lymphocytes include T cells, B cells or natural killer cells. Advantageously, this allows cells which are difficult to be labelled due to their inherently low uptake of foreign particles to be labelled in an efficient and low-cost manner, and allows these cells to be studied in a wider variety of ways and allows for more information to be obtained on these cells.

In an embodiment, the particles are magnetic microparticles or magnetic nanoparticles, preferably magnetic nanoparticles with a hydrodynamic size from 10 nm to 500 nm. The magnetic particles may be used with the centrifuge and magnetic methods, but it would be most advantageous to use the magnetic method to utilise the magnetic properties of the magnetic particles.

The methods described herein may be practised with an apparatus for penetrating a cell with a magnetic particle. The apparatus comprises a vessel having a bottom end for containing a layer of cells at the bottom end; and a force exerting component configured to apply a force to move a plurality of magnetic particles within the vessel towards the bottom end of the vessel to penetrate at least some of the cells with at least some of the plurality of magnetic particles.

Preferably, the bottom end is tapered, preferably the bottom end has a U-shaped or a V-shaped vertical cross section.

In an embodiment, the force exerting component comprises a magnetic array having a plurality of magnets in an alternating polarity arrangement, the magnetic array being configured to apply a magnetic force to pull the magnetic particles within the vessel towards the bottom end of the vessel to penetrate some of the cells with at least some of the magnetic particles.

Preferably, the magnetic array is placed below the tapered bottom end of the vessel to apply the magnetic force, the magnetic force being exerted by a magnetic field that increases from a top region of the vessel to the bottom end of the vessel, wherein the top region is opposite to the tapered bottom end. Preferably, the magnetic field increases exponentially from the top region of the vessel to the bottom end of the vessel, wherein the top region is opposite to the bottom end.

In an embodiment, the magnetic array is configured to generate a significant magnetic force in three axes that concentrates the magnetic particles towards the bottom end of the vessel, wherein one of the three axes is parallel to a longitudinal axis of the vessel, and the three axes are spatially orthogonal to one another. Preferably, the magnetic field that increases from the top region of the vessel to the bottom end of the vessel is greater than 5 milliTesla per millimetre.

In an embodiment, the magnetic force is applied by one magnet of the plurality of magnets.

In an embodiment, the vessel is a well of a multi-well plate, preferably a well of a 96-well plate. Preferably, the magnetic array may also be placed in a plate with the same number of wells. In an embodiment, the plate for the cells have a tapered bottom end whereas the plate for the magnetic array has a flat bottom end. Advantageously, this approach utilises widely available 96 well plates that accommodate both suspension and adherent cells which allow for high-throughput screening at low costs.

In the context of various embodiments, a magnetic particle is any particle that has magnetic properties when subjected to an external magnetic field, and the term “delivery vehicle” refers to a compound or carrier other than the particles as the particles may potentially be attached to another compound or encapsulated within a carrier to bring the particles into the cell.

The magnetic particles described herein may be magnetic microparticles or magnetic nanoparticles. Magnetic microparticles refer to magnetic particles of diameter greater than 1 micron. Magnetic microparticles that are not taken up within the cell may still label the cell by being attached to the surface of the cell. An example is iron oxide particles, which may be contained within a shell, for example a dextran shell. The shell may be further functionalised with a cell penetrating peptide and/or fluorescent dye.

In the context of various embodiments, cells that have high or moderate (in other words not low) intake of the particles includes phagocytic cells and immortalized cell lines. The primary function of phagocytic cells is to uptake foreign material, and can increase in size to uptake bigger targets. Phagocytic cells have increased rates of membrane uptake activity and likely have high intake of the particles. Some immortalized cancer cell lines may be considered to have moderate (elevated uptake compared to low uptake cells) uptake of particles as they typically have overexpression of membrane receptors and higher metabolic activity. Cells that that have low uptake of the particles includes all other cells, and may have low metabolic activity, small cytoplasmic volume and be in a quiescent state. These low uptake cells may be difficult to label as their cellular membrane is not in a state to actively uptake foreign material.

Examples of cells with low uptake of particles include lymphocytes, neutrophils, monocytes, macrophages and stem cells. Stem cells to be labelled typically include stem cells that are administered from an in vitro stage into patients or animals for any form of therapy. The stem cells may be embryonic stem cells, tissue-specific stem cells, mesenchymal stem cells or induced pluripotent stem cells. Examples of tissue-specific or adult stem cells include neural stem cells, hematopoietic stem cells (blood stem cells), mesenchymal stem cells, skin stem cells and epithelial stem cells.

The embodiments described herein may be used as “personalised medicine” in the treatment of patients. This is in the context that many of the immune cell or stem cell therapies, and even theranostics (are possible applications for the described cellular labelling method) are extremely personalised to the patient. Generally in these treatments, the patient's own cells are harvested, “produced” at some scale in vitro, before being returned to the patient. For theranostics, it is personalised as it is first determined which therapeutic the patient responds to (responder vs non-responder).

The custom designed magnetic plate device and/or centrifuge protocol is able to significantly improve the magnetic labelling efficiency of cells-of-interest, such as immune cells intended for adoptive cell transfer immunotherapy. The label time is also greatly shortened compared to existing protocols. The methods are non-vehicular, and do not rely on antibody or liposomal targeting and delivery methods which increase cost and complexity of labelling. Hence, the described methods may be done at a low cost and with a simple procedure.

A key differentiating concept of the described approach herein from existing methods is the use of a sandwich arrangement with target cells placed in the middle. The magnetic nanoparticles (i.e. the label) are inserted into the cells magnetically or centrifugally. Other than concentrating the cells and label into a smaller volume, the additional magneto-mechanical forces on the label (nanoparticle) enable it to penetrate the cell membrane. The centrifugal force contributes to the penetration of the cell, but depending on the relative density of the magnetic label to cell density the felt centrifugal force may be relatively low. To improve the force intensity, magnetism is added as this pulls upon the magnetic label while not affecting the non-magnetic cells. To achieve very high magnetic forces, a custom-designed gradient magnet array is utilised. A single large magnet is not suitable as it would be expensive and potentially dangerous due to the strong magnetic fields generated. Each “magnetic focal point” has to be aligned to the bottom of each well in, for example, a 96 well plate, and the resultant magnetic gradients are much steeper than that of a single magnet, resulting in order-of-magnitude increased magnetic forces (i.e. an exponential increase). The arrangement of the gradient magnetic array results in a safe magnetic field that only works at close range (to the extent of 96 well plate dimensions) and therefore is much safer for researchers. The magnetic field exerts a magnetic force that increases from a top region of the well to the bottom end of the well, thus forming a magnetic field gradient. The magnetic focal point design as opposed to a generic vertical force significantly improves the labelling efficiency of cells with magnetic particles. When combined with a tapered bottom end in the well, the labelling efficiency significantly improves. The significant magnetic force in three axes of the magnetic focal point greatly increases the accumulation of the magnetic particles especially with a tapered bottom end and leads to increased labelling efficiency.

FIG. 1 shows a schematic of the magnetic labelling of cells. In the top, magnetic particles, more specifically, magnetic nanoparticles (MNPs), for example 25 nm nanoparticles, are to be inserted into cells. In an existing method shown on the left, the magnetic nanoparticles are passively incubated with the cells for a certain amount of time. However, this is inefficient as the time required is low with poor uptake by the cells. In the method described herein, the magnetic nanoparticles are inserted into the cells in a short amount of time and with high uptake with magneto-mechanical forces from the magnetic forces (indicated by arrow 15) and/or centrifugal forces (indicated by arrow 20). Either force may be used by itself and may also be used in combination. The centrifugal forces (arrow 20) are applied first to layer the cells at the bottom of the V-shaped or U-shaped cross section of the well 5. Subsequently, magnetic forces (arrow 15) are applied to form a sandwich of the cells between the magnetic label and the bottom of the cell and enable the magnetic labels to gradually penetrate into the sandwiched cells as shown in the right of FIG. 1 where the big circles representing the cells are between the small circles representing the magnetic labels and the bottom of the well 5.

An example of magnetic particles that may be used include magnetic nanoparticles composed of iron oxide particles contained within a dextran shell. While iron oxide is typically used, any particle with a diameter smaller than the targeted cells (including microparticles and nanoparticles) and with magnetic properties that experiences a motive force under a magnetic gradient field may be used. The dextran shell may be further functionalised by the conjugation of cell penetrating peptides as described or by conjugation with a fluorescent dye to enable optical tracking of label uptake.

### Example 1—Centrifuge Sandwich Method

The cells-to-be-labelled are first dispensed into each of the wells 5 in a 96-well plate at an optimal amount to ensure a dense cellular monolayer. Multiple layers are detrimental as upper layers will shield the bottom layers of cells preventing the labelling. For a suspension of cells, a quick centrifuge pulse is first performed to ensure all the cells are flushed to the bottom of the well 5 ideally forming a monolayer. Magnetic nanoparticles are then added to the culture media in the wells 5. Subsequently a 3 hour spin protocol at 830 rcf and 32 degrees Celsius is performed to generate centrifugal force on the magnetic nanoparticles which migrate towards to the bottom of the well 5. The centrifugal force not only concentrates the nanoparticles close to the cell monolayer, but as a result of the sandwich orientation, the downwards force (arrow 20 in FIG. 1) aids the mechanical penetration of the magnetic nanoparticles through the cell membrane as the density of the magnetic nanoparticles is higher than the density of the cells. The magnitude of the force depends on the relative density of the magnetic nanoparticles to that of the target cell. This may be difficult to increase beyond a certain point because the cells can only take a maximal amount of g-force and also because the density of the iron oxide (magnetite) cannot be increased beyond that allowed by crystalline structure.

### Example 2—Magnetic Sandwich Method

The cells-to-be-labelled are first dispensed into each well 5 in a 96-well plate at an optimal amount to ensure a dense cellular monolayer. Multiple layers are detrimental as upper layers will shield bottom layers of cells. For a suspension of cells, a quick centrifuge pulse is first performed to ensure all the cells are flushed to the bottom of the well 5 ideally forming a monolayer. Magnetic nanoparticles are then added to the culture media in the wells 5 in RPMI 1640 (Roswell Park Memorial Institute 1640) medium and 0.5% v/v Fetal Bovine Serum (FBS) without addition of ethylenediaminetetraacetic acid (EDTA). The magnetic nanoparticles used may be iron oxide particles contained in a dextran shell. The absence of EDTA is unusual as EDTA is commonly added to magnetic nanoparticles suspensions to prevent clumping, but the presence of EDTA during the labelling reduces cell viability. The 0.5% v/v of FBS was determined to be optimum as increasing the FBS concentration impedes labelling efficiency and decreasing the FBS concentration reduces cell viability.

Subsequently, the 96 well plate with cells is placed on a specialised magnetic plate 25 (the alignment of the wells to plate focal points is important as described below) and put together into the incubator at 37 degrees Celsius. An optimal duration for the magnetic labelling is between 2 to 3 hours depending on the cell type. It was found that shorter incubation times yield reduced labelling efficiencies while longer incubation times result in a loss of cell viability.

The magnetic gradients generated by permanent magnets 35 in the magnetic plate 25 generate or exert a magnetic force that causes the nanoparticles to migrate to the bottom of the well 5. This force not only concentrates the nanoparticles close to the cell monolayer, but as a result of the sandwich orientation, the downwards force aids the mechanical penetration of the magnetic nanoparticles through the cell membrane of the cellular monolayer sandwich between the magnetic nanoparticles and well bottom as the magnetic nanoparticles have magnetism while the cells do not, as shown in the right of FIG. 1. Furthermore, the magnetic field increases exponentially as the magnet 35 is approached, leading to increased magnetic force when the nanoparticles are on or in proximity to the cell monolayer at the very bottom of the well 5, further increasing the magnetic force felt to ensure entry of the magnetic nanoparticles into the cell even without the aid of cell penetrating peptides. The magnitude of the force depends on the saturation magnetisation and magnetic mass of the nanoparticles as well as the magnetic gradient strength. This is easier to increase as static magnetic fields have no negative effects on the cells even when increased to very high levels. Larger nanoparticles with more magnetic mass would also increase this force, but this must be balanced against harder uptake by the cells for physically larger particles.

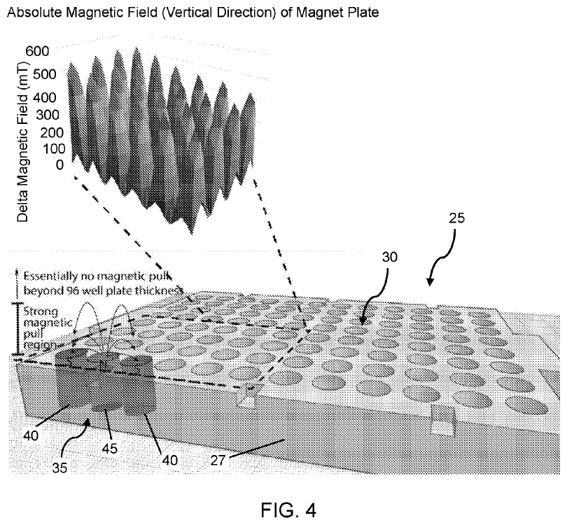

The magnetic plate 25 or array used in Example 2 is made with specific features for the use in the magnetic sandwich method. The magnetic plate 25 has a plurality of magnets 35 and each are aligned to the exact centre of each of the U or V points (i.e. the lowest point or bottom) of each well 5 in the 96 well plate. In an example, the magnetic plate 25 may have a base structure 27 with a plurality of holes 30 on one side of the base structure 27 as shown in FIG. 4. The magnet 35 may be placed within each hole 30, this has the advantage of holding the magnet 35 in place. For example, a 96-well plate with cylindrical wells 30 having flat bottoms may be used. Advantageously, when the wells (or holes) 30 and the magnets 35 are cylindrical, they fit snuggly and provide a convenient way of storing and transporting the magnets 35. Other shapes of the holes 30 and/or magnets 35 may be used whilst still utilising the concept described herein. A detachable cover may be provided as well. The base structure 27 and detachable cover should be made of non-magnetic materials. It should be noted that while it may be convenient for the magnetic plate 25 and the plate for the labelling of the cells to be of the same dimension and have the same number of wells, this is merely for convenience and is not necessary as long as the magnets 35 can be positioned as described herein.

To increase the magnetic gradient to the maximum, an alternating polarity strategy is used as shown in FIG. 4 where the polarity of the magnets 35 alternate between adjacent magnets in the magnetic plate. FIG. 4 shows three magnets 35 with alternating north pole 40 and south pole 45 at the bottom end of the magnets 35. This is different from just a simple block magnet which has a single polarity. Advantageously, this design ensures that there is maximum vertical force only at the centre of the well 5 where the cells are of the highest density, thereby increasing the efficiency of the magnetic sandwich effect. The flux lines align horizontal in between opposite polarity neighbouring magnets 35, therefore ensuring that the magnetic nanoparticles do not vertically deposit at the sides of the well 5 where there are few cells. This translates to little wastage of the magnetic nanoparticles. Further, the configuration of the magnetic plate 25 maximises the change in vertically oriented magnetic field strength with distance from the magnet 35, which translates directly to maximised magnetic force directed (vertically) into the sandwiched cells at the bottom of the well 5. The flux lines originating from the centre of the magnet 35 are redirected to neighbouring opposite polarity magnets 35 and not allowed to spread out into space as is the case for a single polarity plate magnet. Further, the alternating magnet polarity minimises stray flux lines beyond the close vicinity of the plate surface, increasing safety and ease-of-handling as magnetic objects beyond the thickness of the 96 well plate are unaffected by the magnetic array 25. The top part of FIG. 4 illustrates a three-dimensional graph of the magnetic field to illustrate how the magnetic field strength is focused at the centre of the well 5 containing the cells and weakens as the distance from the magnet 35 increases. This is important as magnetic fields can affect other electrical equipment, especially in a medical setting, and it is necessary to ensure the magnetic array 25 does not unintentionally disrupt other equipment.

As seen in FIG. 4, the design of the magnetic array 25 is specifically optimised for the magnet sandwich mechanism as it maximizes vertical pull force at the centre of the U-shaped or V-shaped bottom of each of the 96 wells 5 and minimises vertical pull force everywhere else, ensuring that nanoparticles are not wasted on other regions of the well 5 with lower cell density. The field lines at the edge of the well 5 are designed to flow horizontally such that nanoparticles are not pulled to the edges of the well 5 but rather to the centre of the well 5. Lastly, the design maximizes safety as there is only a strong magnetic pull force within 1 to 2 cm of the plate and there is essentially no pull beyond this distance due to the redirection of flux lines into neighbouring magnets rather than allowing them to extend out into space as is the case of a single-polarity magnet. While alternating arrays of magnets 35 may not be completely new, the specific design herein is optimized for the use on 96-well plates which commonly used in biology experiments. Shaping the magnetic directionality of the pull forces within each of the 96 wells 5 is a key differentiator that enables high efficiency (isocentre labelling) as opposed to general-direction pull forces of other magnets (single polarity array or other arrays).

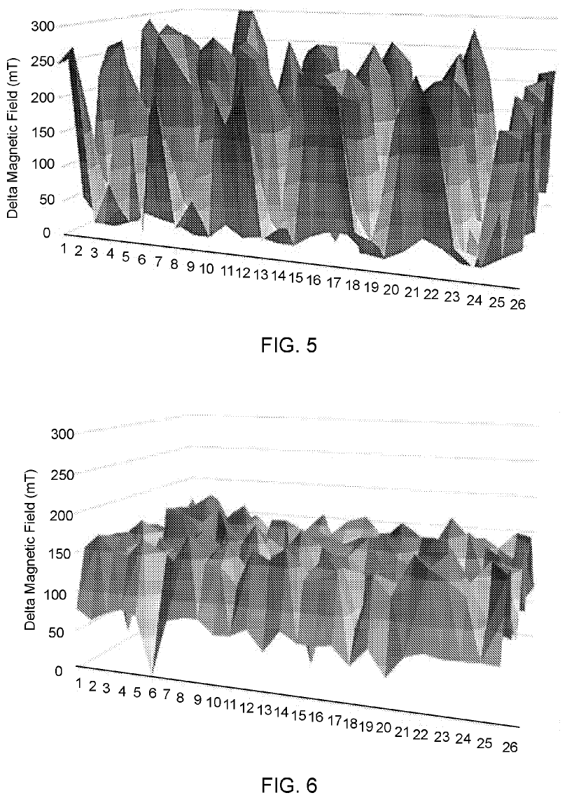

FIG. 5 shows the magnetic foci at each well centre and has a magnetic concentration factor in the 3-dimension axes of about 1000. FIG. 6 shows the magnetic strength of a commercial magnet slab used for cell separation. It may be seen that the magnet slab has weak uniform pull with no magnetic focus. The magnetic concentration factor is only in one direction (z-axis).

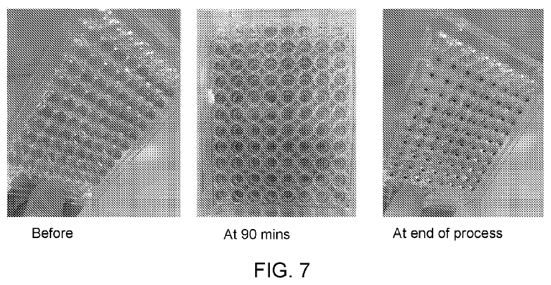

FIG. 7 shows three pictures of the cells being labelled with the magnetic nanoparticles. In FIG. 7, from the left to right, the pictures show the cells in the 96-well plate before the start of the magnetic sandwich process, after 90 mins, and at the end of the process respectively. At 90 mins and the end of the process, a red dot may be observed at the end of the plate indicating the concentration of a solid mass.

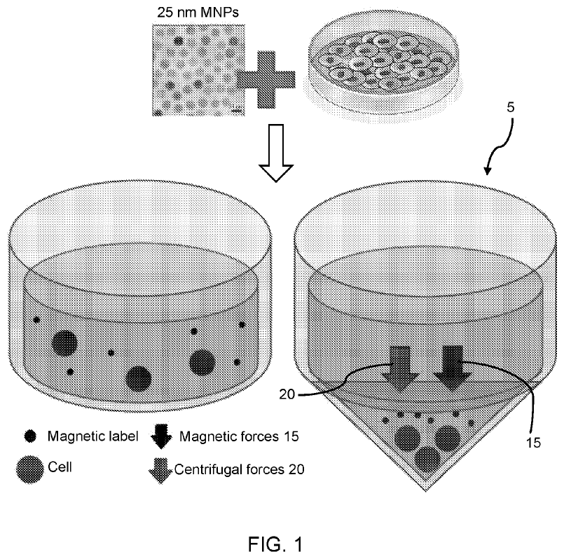

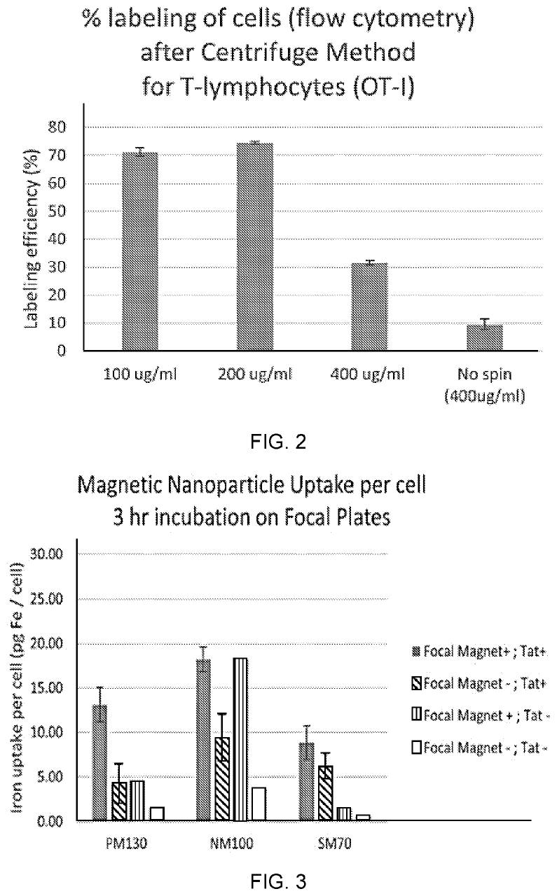

The labelling results show that the use of centrifuge method or magnetic plate (FIG. 2 and FIG. 3) method both significantly increase the amount of iron uptake into the immune cell (T-lymphocytes). This is regardless of the presence or absence of cell penetrating peptides such as Tat, although adding Tat does further enhance the uptake. The method also works with a variety of nanoparticles (PM130, NM100 and SM70) of different hydrodynamic sizes (130 nm, 100 nm and 70 nm respectively). FIG. 2 shows the magnetic nanoparticle uptake efficiency for the centrifuge sandwich method in Example 1 with a 1.5-2.5 hour spin. The results show that increasing nanoparticle concentration in media does not improve the labelling efficiency where a concentration of 100 μg/ml and 200 μg/ml magnetic nanoparticles have a labelling efficiency of 70% and higher, which drops to 30% when the concentration increases to 400 μg/ml. The data suggests that centrifugation causes migration of nanoparticles towards the bottom of well where it reaches a much higher effective concentration that plateaus and thus any further increases in the media concentration of nanoparticles has no effect and may be detrimental. However, it should be noted that the centrifuge sandwich method increases the labelling efficiency by at least 3 times which is a significant increase.

FIG. 3 shows the magnetic nanoparticle uptake efficiency for the Focal Magnetic Plate sandwich method with a 3 hour incubation. The results show that the Focal Magnet plate increases the uptake significantly over the Tat peptide alone, and thus further improves the uptake above what Tat peptide performs.

Advantageously, the methods and apparatuses described herein allow for a lower concentration of particles including magnetic nanoparticles to be used in the labelling leading to significantly less wastage of the particles. This avoids the wastage problem with existing methods where a high concentration of particles like nanoparticles are required for high labelling efficiency leading to large portion of the particles like nanoparticles remaining behind in the solution media. The method and apparatus described herein effectively achieves high concentrations with low amounts of nanoparticles.

Advantageous, the methods and apparatuses described herein allow cells with low innate intake of foreign particles to be labelled in an efficient and low-cost manner, which is amenable to high throughput labelling. Hence, it allows these difficult to label cells to be studied more widely and expands the knowledge on these cells.

1. A method of penetrating a cell with a particle, the method comprising:
(a) providing a layer of cells at or proximal to a bottom end of a vessel;
(b) adding a plurality of particles to the vessel; and
(c) applying a force to move the plurality of particles towards the bottom end of the vessel to cause at least some of the plurality of particles to penetrate at least some of the cells.

2. The method according to claim 1, wherein the bottom end of the vessel is tapered, preferably the bottom end has a U-shaped or a V-shaped vertical cross-section.

3. The method according to claim 1, wherein the force comprises a magnetic force exerted by a magnetic field that increases from a top region of the vessel to the bottom end of the vessel, the top region being opposite to the bottom end, and wherein the plurality of particles are magnetic particles.

4. The method according to claim 3, wherein the magnetic field increases exponentially from the top region of the vessel to the bottom end of the vessel.

5. The method according to claim 3, wherein the magnetic force is a significant magnetic force in three axes that concentrates the plurality of particles towards the bottom end of the vessel, wherein one of the three axes is parallel to a longitudinal axis of the vessel, and the three axes are spatially orthogonal to one another, preferably the magnetic field that increases from the top region of the vessel to the bottom end of the vessel is greater than 5 milliTesla per millimetre; or wherein the magnetic force is generated by one magnet of a plurality of magnets in a magnetic array, wherein the plurality of magnets is arranged in an alternating polarity configuration.

6. (canceled)

7. The method according to claim 3, wherein at least one of the following conditions are fulfilled:
(i) the magnetic force is applied for at least 5 minutes, preferably at most 12 hours, more preferably from 2 to 3 hours; and
(ii) the magnetic force is applied in the absence of ethylenediaminetetraacetic acid or a salt thereof, or
(iii) wherein when the plurality of particles comprises iron oxide particles, or preferably iron oxide particles contained within a dextran shell, the method provides an iron uptake of at least 1 picogram iron per cell and/or the plurality of particles is present at a concentration of at least 200 μg per mL.

8-9. (canceled)

10. The method according to claim 3, wherein the force comprises a centrifugal force in combination with the magnetic force, the centrifugal force being applied to centrifuge the cells for at least 1 hour, preferably at most 3 hours.

11. The method according to claim 1, wherein the force comprises a centrifugal force, the centrifugal force being applied to centrifuge the cells for at least 1 hour, preferably at most 3 hours.

12. (canceled)

13. The method according to claim 1, wherein the layer of cells is present at a concentration of at least 100,000 cells per vessel;
and/or wherein the layer is a single layer only, preferably a dense cellular single layer; and/or wherein the particles have higher density than the cells, preferably the particles have at least 10 times higher density than the cells, more preferably the particles have at least 50 times higher density than the cells, even more preferably the particles have at least 100 times higher density than the cells; and/or wherein the particles are magnetic microparticles or magnetic nanoparticles, preferably magnetic nanoparticles with a hydrodynamic size from 10 nm to 500 nm.

14-15. (canceled)

16. The method according to claim 1, wherein providing the layer of cells at or proximal to the bottom end of the vessel comprises centrifuging the cells to provide the layer of cells at or proximal to the bottom end of the vessel.

17. (canceled)

18. The method according to claim 1, comprising adding a delivery vehicle to increase the penetration of the particles into the cells, wherein the delivery vehicle is selected from the group consisting of antibodies, liposomes, ligands and cell-penetrating peptides.

19. The method according to claim 18, wherein the delivery vehicle is a cell-penetrating peptide, preferably the cell-penetrating peptide is a Tat peptide, wherein the Tat peptide comprises SEQ ID NO: 1 and a linker, the linker comprising any suitable functional groups that allow linking and conjugation, preferably the linker comprises amino acids.

20. (canceled)

21. The method according to claim 1, wherein a delivery vehicle is absent from the vessel.

22. The method according to claim 1, wherein the cells have an innate low uptake of the particles, preferably the cells are selected from the group consisting of lymphocytes, neutrophils, monocytes, macrophages and stem cells, preferably wherein the lymphocytes include T cells, B cells or natural killer cells.

23-24. (canceled)

25. An apparatus for penetrating a cell with a magnetic particle, the apparatus comprising: a vessel having a bottom end for containing a layer of cells at the bottom end; and a force exerting component configured to apply a force to move a plurality of magnetic particles within the vessel towards the bottom end of the vessel to penetrate at least some of the cells with at least some of the plurality of magnetic particles.

26. The apparatus according to claim 25, wherein the bottom end is tapered, preferably the bottom end has a U-shaped or a V-shaped vertical cross section.

27. The apparatus according to claim 25, wherein the force exerting component comprises a magnetic array having a plurality of magnets in an alternating polarity arrangement, the magnetic array being configured to apply a magnetic force to pull the magnetic particles within the vessel towards the bottom end of the vessel to penetrate some of the cells with at least some of the magnetic particles.

28. The apparatus according to claim 27, wherein the magnetic array is placed below the bottom end of the vessel to apply the magnetic force, the magnetic force being exerted by a magnetic field that increases from a top region of the vessel to the bottom end of the vessel, wherein the top region is opposite to the bottom end; and/or wherein the magnetic array is configured to generate a significant magnetic force in three axes that concentrates the magnetic particles towards the bottom end of the vessel, wherein one of the three axes is parallel to a longitudinal axis of the vessel, and the three axes are spatially orthogonal to one another, preferably the magnetic field that increases from the top region of the vessel to the bottom end of the vessel is greater than 5 milliTesla per millimetre.

29-30. (canceled)

31. The apparatus according to claim 27, wherein the magnetic force is applied by one magnet of the plurality of magnets.

32. The apparatus according to claim 25, wherein the vessel is a well of a multi-well plate, preferably a well of a 96-well plate.
